(19) United States
(12) Patent Application Publication
(10) Pub. No.: US 2025/0361265 A1
(43) Pub. Date: Nov. 27, 2025

(54) MIXED MODE SUBTRACTIVE ANION EXCHANGE CHROMATOGRAPHY LIGANDS BASED ON 4-(2-(DIMETHYLAMINO)ETHOXY)ANILINE STRUCTURES

(71) Applicant: BIO-RAD LABORATORIES, INC., HERCULES, CA (US)

(72) Inventors: CHRISTOPHER BELISLE, WALNUT CREEK, CA (US); JIA-LI LIAO, SAN RAMON, CA (US)

(21) Appl. No.: 18/671,086

(22) Filed: May 22, 2024

Related U.S. Application Data
(60) Provisional application No. 63/503,496, filed on May 22, 2023.

(51) Int. Cl. C07K 1/16; B01D 15/36; B01D 15/38; B01J 20/22; B01J 20/289; B01J 20/32; C07C 217/84

(52) U.S. Cl. CPC C07K 1/165; B01D 15/363; B01D 15/3847; B01J 20/22; B01J 20/289; B01J 20/321; B01J 20/3219; B01J 20/3261; B01J 20/3293; C07C 217/84; B01J 2220/52

(57) ABSTRACT

This disclosure pertains to mixed mode chromatography ligands and chromatography matrices suitable for the purification of proteins from biological sources or biological samples. Methods of making chromatography matrices comprising the disclosed ligands are also disclosed. Similarly, methods of purifying proteins from a biological sample, source solution, or source liquid using the disclosed chromatography matrices are also provided.

### CROSS-REFERENCE TO RELATED APPLICATION

This application claims the benefit of U.S. Provisional Application Ser. No. 63/503,496, filed May 22, 2023, the disclosure of which is hereby incorporated by reference in its entirety, including all figures, tables and amino acid or nucleic acid sequences.

### FIELD OF THE INVENTION

Materials and methods for separating immunoglobulins or other proteins from source liquids, for purposes of purification or isolation, utilizing chromatographic separation techniques are provided. Methods of preparing chromatographic materials suitable for use in such techniques are also provided.

### BACKGROUND

The separation of proteins, such as immunoglobulins or other therapeutic biological agents, from source liquids, such as mammalian bodily fluids or cell culture harvest or supernatants, is of significant commercial interest and value. Also of interest are preparations of proteins in a sufficiently concentrated or purified form for diagnostic, laboratory, and therapeutic uses. However, the purification of proteins often suffers from factors such as low yield, the use of costly separation media (chromatography media), the leaching of separation media (for example, chromatography ligands) into the product, and concerns for the safe disposal of extraneous materials used in the extraction process. The present invention seeks to address at least some of these issues.

### BRIEF SUMMARY

This disclosure provides mixed mode chromatography ligands and chromatography matrices suitable for the purification of proteins from biological sources or samples. Methods of making chromatography matrices and using the disclosed chromatography ligands are also provided.

### BRIEF DESCRIPTION OF THE DRAWINGS

FIGS. 1A-1B. Synthesis diagrams for exemplary ligands (FIG. 1A CB466q, FIG. 1B, CB466x).

FIG. 2. Elution profile for CB466x.

FIG. 3. Elution profile for CB466q.

FIG. 4. Elution profile for CB464b4.

FIG. 5. Elution profile for CB467b.

FIG. 6. Elution profile for Capto Q ImpRes.

FIG. 7. Elution profile for Nuvia aPrime 4A.

### DETAILED DISCLOSURE OF THE INVENTION

Unless otherwise stated, the following terms used in this application, including the specification and claims, have the definitions given below.

As used herein, the singular forms “a”, “an” and “the” are intended to include the plural forms as well, unless the context clearly indicates otherwise. Furthermore, to the extent that the terms “containing”, “including”, “includes”, “having”, “has”, “with”, or grammatical variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably. Definition of standard chemistry terms can be found in reference works, including Carey and Sundberg (2007) “Advanced Organic Chemistry 5th Ed.” Vols. A and B, Springer Science+Business Media LLC, New York. The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology.

The term “and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z).

The phrases “consisting essentially of” or “consists essentially of” indicate that the claim encompasses embodiments containing the specified materials or steps and those that do not materially affect the basic and novel characteristic(s) of the claim.

The term “about” or “approximately” means within an acceptable error range for the particular value as determined by one of ordinary skill in the art, which will depend in part on how the value is measured or determined, i.e., the limitations of the measurement system. The terms “about” and “approximately” are meant to encompass a range of +20%, +10% or +5% of a given value. Thus, in the context of compositions containing amounts of ingredients where the terms “about” or “approximately” are used, these compositions can contain the stated amount of the ingredient with a variation (error range) of 0-10% around the value (X±10%). In the context of pH, the term “about” or “approximately” encompasses a range of ±0.2 units of a given value.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The terms “biological sample(s)”, “source solution(s)”, or “source liquid(s)” refer to any composition containing a target molecule of biological origin (a “biomolecule”) that is desired to be purified. Non-limiting examples of target molecules include: antibodies, enzymes, growth regulators, clotting factors, transcription factors and phosphoproteins. In some embodiments, the target molecule (biomolecule) to be purified is an antibody or a non-antibody protein. Non-limiting examples of biological samples include serum samples from individuals or cell culture supernatants (e.g., clarified cell culture supernatants). With respect to the purification of biomolecules, such as antibodies, any biological sample that contains the target biomolecule can be used. Non-limiting examples of a source solution or source liquid include unpurified or partially purified antibodies from natural, synthetic, or recombinant sources.

Unpurified antibody preparations (source solutions) can come from various sources including, but not limited to, plasma, serum, ascites, milk, plant extracts, bacterial lysates, yeast lysates, or conditioned cell culture media. Partially purified antibody preparations can come from unpurified preparations that have been processed by at least one chromatography, precipitation, other fractionation step, or any combination of the foregoing. In some embodiments, the antibodies have not been purified by protein A affinity prior to purification. Other embodiments utilize antibody preparations that have undergone a preliminary affinity purification step utilizing protein A or protein G.

“Antibody” refers to an immunoglobulin, composite (e.g., fusion protein), or fragmentary form thereof. The term includes but is not limited to polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” also includes composite forms including but not limited to fusion proteins containing an immunoglobulin moiety. “Antibody” also includes antibody fragments such as Fab, F(ab′)₂, Fv, scFv, Fd, dAb, Fc, whether or not they retain antigen-binding function.

The term “protein” refers to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers (e.g., recombinant proteins).

“Bind-elute mode” refers to an operational approach to chromatography in which the buffer conditions are established so that target molecules and, optionally undesired contaminants, bind to the ligand when the sample is applied to the ligand. Fractionation of the target can be achieved subsequently by changing the conditions such that the target is eluted from the support. In some embodiments, contaminants remain bound following target elution. In some embodiments, contaminants either flow-through or are bound and eluted before elution of the target.

“Flow-through mode” refers to an operational approach to chromatography in which the buffer conditions are established so that the target molecule to be purified flows through the chromatography support comprising the ligand, while at least some sample contaminants are selectively retained, thus achieving their removal from the sample.

The terms “matrix” or “support matrix” can be used interchangeably. In various embodiments, the matrix can be particles, a membrane or a monolith, and by “monolith” is meant a single block, pellet, or slab of material. Particles when used as matrices can be spheres or beads, either smooth-surfaced or with a rough or textured surface. Many, and in some cases all, of the pores are through-pores, extending through the particles to serve as channels large enough to permit hydrodynamic flow or fast diffusion through the pores. When in the form of spheres or beads, the median particle diameter, where the term “diameter” refers to the longest exterior dimension of the particle, is preferably within the range of about 25 microns to about 150 microns. The spheres or beads can have pores of a median diameter of 0.5 micron or greater, optionally with substantially no pores of less than 0.1 micron in diameter. In certain embodiments of the invention, the median pore diameter ranges from about 0.5 micron to about 2.0 microns. The pore volume can vary, although in many embodiments, the pore volume will range from about 0.5 to about 2.0 cc/g. Disclosures of matrices meeting the descriptions in this paragraph and the processes by which they are made are found in Hjerten et al., U.S. Pat. No. 5,645,717, Liao et al., U.S. Pat. No. 5,647,979, Liao et al., U.S. Pat. No. 5,935,429, and Liao et al., U.S. Pat. No. 6,423,666. Examples of monomers that can be polymerized to achieve useful matrices are vinyl acetate, vinyl propylamine, acrylic acid, methacrylate, butyl acrylate, acrylamide, methacrylamide, vinyl pyrrolidone (vinyl pyrrolidinone), with functional groups in some cases. Crosslinking agents are also of use in many embodiments, and when present will generally constitute a mole ratio of from about 0.1 to about 0.7 relative to total monomer. Examples of crosslinking agents are dihydroxyethylenebisacrylamide, diallyltartardiamide, triallyl citric triamide, ethylene diacrylate, bisacrylylcystamine, N,N′-methylenebisacrylamide, 20 and piperazine diacrylamide.

The chromatography ligands are linked to the chromatography matrix via a linker to form a “chromatography resin” or “chromatography matrix”. Linkage of the chromatography ligand to the matrix will depend on the specific matrix used and the chemical group to be linked to the matrix. Ligands can be linked to the matrix by performing a reaction between the ligand, for example and amine group, and a functional group on the matrix, for example, an aldehyde or diol group. For matrices that do not have a suitable functional group, the matrix is reacted with a suitable activating reagent to create a suitable functional group to which the chromatography ligand can be attached.

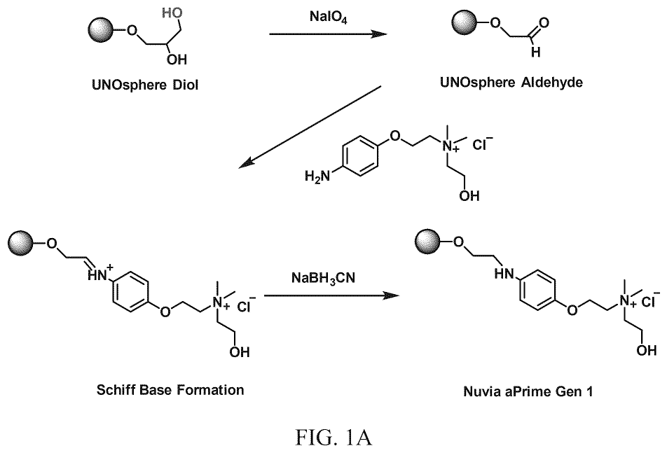

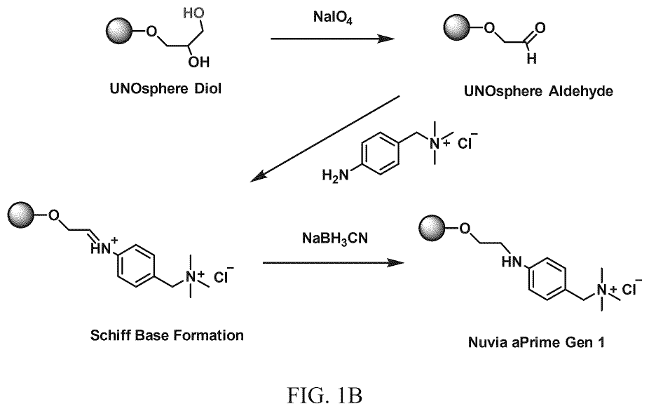

For purposes of the formation of a linkage with the chromatography ligand, the inclusion of monomers with vicinal diols attached to the matrix is useful. One monomer example is allyloxy propandiol (3-allyloxy-1,2-propanediol). Vicinal diol monomers can be used with other monomers to prepare copolymers. The diol group density in the polymers produced from diol-containing monomers can vary widely, such as for example densities within a range of from about 100 to 1,000 μmol/mL (i.e., micromoles of diol per milliliter of packed beads), and in many cases a range of from about 200 to 300 μmol/mL. An example of a matrix that meets this description and is commercially available is UNOsphere™ Diol (Bio-Rad Laboratories, Inc., Hercules, Calif., USA). To couple a pendant amine-containing ligand to a matrix with exposed vicinal diols, the diols can be oxidized to aldehyde groups, and the aldehyde groups can then be coupled to amine groups to form secondary amino linkages, all by conventional chemistry techniques well known in the art. In some embodiments, the matrix comprises a diol, which is converted to an aldehyde, e.g., by conversion with NaIO₄. The primary amine of the ligand can be linked to an aldehyde on the matrix by a reductive amination reaction by the scheme provided in Example 1 and FIGS. 1A-1B.

As used herein, the term “linker” refers to a molecule having 1-10 carbon atoms, preferably an alkyl group. The linker has a neutral charge and can include cyclic groups. The linker links the chromatographic ligand to the chromatography matrix.

As used herein, the term “alkyl” refers to a straight or branched, saturated, aliphatic radical having between 1-10 carbon atoms. For example, C₁-C₆ alkyl includes, but is not limited to, methyl, ethyl, propyl, isopropyl, butyl, isobutyl, sec-butyl, tert-butyl, pentyl, isopentyl, and/or hexyl. The alkyl can include any number of carbons, such as 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, 9-10, or 10 carbons. The alkyl group is typically monovalent, but can be divalent, such as when the alkyl group links two chemical groups together. This disclosure provides a variety of chromatography ligands, described in Table 1. As would be apparent to those skilled in the art, the linker attaching the ligand to the solid support (matrix) can be an alkyl group between 1 and 10 carbons in length, preferably between 1 and 5 carbons in length, or 1 to 3 carbons in length (the sphere and line connected to the ligand representing the matrix and linker as illustrated below).

This disclosure provides a number of novel ligands suitable for use in mixed mode anion exchange chromatography (AEX). The ligands have the general structure:

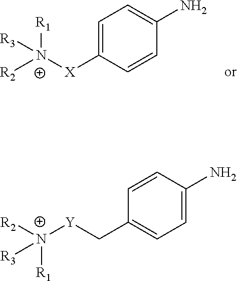

where R₁, R₂, and R₃ can be the same or different and are C₁-C₁₀ alkyl, H, or —(CH₂CH₂O)ₙ—H (ethyloxy), where n is 1, 2, 3, 4, or 5, and at least one of R₁, R₂, and R₃ is not H; X is —(C₁-C₅ alkyl)- or —(CH₂CH₂O)ₙ— (ethyloxy), and n is 1, 2, 3, 4, or 5; and Y is —(C₁-C₅ alkyl)- or (CH₂CH₂O)ₙ— (ethyloxy), and n is 1, 2, 3, 4, or 5, the oxygen of the ethyloxy group in X or Y being bonded to the benzyl ring when present.

In the context of R₁, R₂, and R₃, the term “alkyl” refers to a straight or branched, saturated, aliphatic radical having between 1-10 carbon atoms. In the context of this disclosure, the use of a range for the alkyl groups, such as C₁-C₁₀ alkyl permits for the exclusion of one of more alkyl from the range (for example, if a C₅ alkyl group is to be excluded, the range can be re-written as C₁-C₄ alkyl and C₆-C₁₀ alkyl). Alternatively, the range can be presented as C₁-Cₓ alkyl, where x is an integer between 2 and 10, and includes C₁ alkyl, C₂ alkyl, C₃ alkyl, C₄ alkyl, C₅ alkyl, C₆ alkyl, C₇ alkyl, C₈ alkyl, C₉ alkyl, and C₁₀ alkyl (any of which can be included or excluded within the range recited). Thus, a C₁-C₆ alkyl includes, but is not limited to, methyl, ethyl, propyl, isopropyl, butyl, isobutyl, sec-butyl, tert-butyl, pentyl, isopentyl, and/or hexyl. As discussed above, the C₁-C₁₀ alkyl can include any number of carbons, such as 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, 9-10, or 10 carbons. The alkyl groups of R₁, R₂ and/or R₃ can be substituted by one, two or three radicals independently selected from C₁-C₁₀ alkyl, a benzyl group, a phenyl group, or a hydroxyl group.

In certain embodiments, R₁, R₂, and/or R₃ can be the same or different and are H, C₁-C₅ groups or C₁-C₃ groups, optionally substituted with one or more hydroxyl group with the proviso that at least one of R₁, R₂, and R₃ is not H. In yet other embodiments, one or more of R₁, R₂ or R₃ is —CH₂CH₂OH group and the other radicals can be the same or different and are a H, C₁-C₁₀ alkyl group, a C₁-C₅ alkyl group, a C₁-C₃ alkyl group, a methyl group, an ethyl group, or a propyl group, preferably a C₁-C₁₀ alkyl group, a C₁-C₅ alkyl group, a C₁-C₃ alkyl group, a methyl group, an ethyl group, or a propyl group.

In specific embodiments, these ligands are designated as, CB466x, CB4661, CB464b4 and CB467b. The structures of these ligands is provided in Table 1.

TABLE 1

| | | IC |
|---|---|---|
| Structure | Ligand | (μ mol/mL) |
| 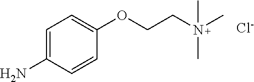 | CB466x | 62 |
| 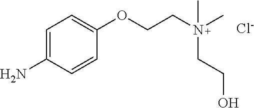 | CB466q | 79 |
| 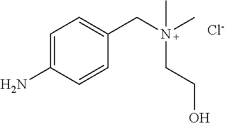 | CB464b4 | 64 |
| 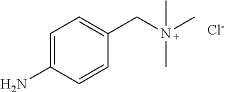 | CB467b | 60 |

The disclosed ligands can be synthesized by standard chemical reactions. These ligands can then be immobilized on a solid support to form a chromatography resin. Additionally, the amine functional group associated with the benzyl group can be provided at the ortho, meta, or para position.

The disclosure also provides a mixed-mode chromatography medium (chromatography resin) comprising a solid support, linker and ligand having the formula:

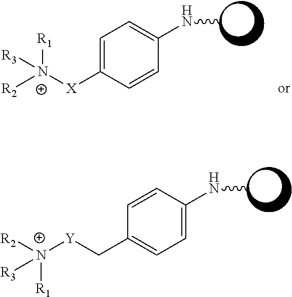

where R₁, R₂, and R₃ can be the same or different and are optionally substituted C₁-C₁₀ alkyl, H, or optionally substituted —(CH₂CH₂O)ₙ—H (ethyloxy), where n is 1, 2, 3, 4, or 5, with the proviso that at least one of R₁, R₂, and R₃ is not H when one of R₁, R₂, and R₃ is H; X is —(C₁-C₈alkyl)- or —(CH₂CH₂O)ₙ— (ethyloxy), and n is 1, 2, 3, 4, or 5; and Y is —(C₁-C₅ alkyl)- or —(CH₂CH₂O)ₙ— (ethyloxy), and n is 1, 2, 3, 4, or 5, the oxygen of the ethyloxy group in X or Y being bonded to the benzyl ring when present. In certain embodiments one or two of R₁, R₂, and R₃ is H and the remaining variables are optionally substituted C₁-C₁₀ alkyl, H, or optionally substituted —(CH₂CH₂O)ₙ—H (ethyloxy), where n is 1, 2, 3, 4, or 5. In other embodiments, R₁, R₂, and R₃ can be the same or different and are optionally substituted C₁-C₁₀ alkyl or optionally substituted —(CH₂CH₂O)ₙ—H (ethyloxy), n is 1, 2, 3, 4, or 5.

In the context of R₁, R₂, and R₃, the term “alkyl” refers to a straight or branched, saturated, aliphatic radical having between 1-10 carbon atoms. In the context of this disclosure, the use of a range for the alkyl groups, such as C₁-C₁₀ alkyl permits for the exclusion of one of more alkyl from the range (for example, if a C₅ alkyl group is to be excluded, the range can be re-written as C₁-C₄ alkyl and C₆-C₁₀ alkyl). Alternatively, the range can be presented as C₁-Cₓ alkyl, where x is an integer between 2 and 10, and includes C₁ alkyl, C₂ alkyl, C₃ alkyl, C₄ alkyl, C₅ alkyl, C₆ alkyl, C₇ alkyl, C₈ alkyl, C₉ alkyl, and C₁₀ alkyl (any of which can be included or excluded within the range recited). Thus, a C₁-C₆ alkyl includes, but is not limited to, methyl, ethyl, propyl, isopropyl, butyl, isobutyl, sec-butyl, tert-butyl, pentyl, isopentyl, and/or hexyl. As discussed above, the C₁-C₁₀ alkyl can include any number of carbons, such as 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, 9-10, or 10 carbons. The alkyl groups of R₁, R₂ and/or R₃ can be substituted by one, two or three radicals independently selected from C₁-C₁₀ alkyl, a benzyl group, a phenyl group, or a hydroxyl group.

In certain embodiments, R₁, R₂, and/or R₃ can be the same or different and are H, C₁-C₅ groups or C₁-C₃ groups, optionally substituted with one or more hydroxyl group with the proviso that at least one of R₁, R₂, and R₃ is not H. In yet other embodiments, one or more of R₁, R₂ or R₃ is —CH₂CH₂OH group and the other radicals can be the same or different and are a H, C₁-C₁₀ alkyl group, a C₁-C₅ alkyl group, a C₁-C₃ alkyl group, a methyl group, an ethyl group, or a propyl group, preferably a C₁-C₁₀ alkyl group, a C₁-C₅ alkyl group, a C₁-C₃ alkyl group, a methyl group, an ethyl group, or a propyl group.

Protein purification utilizing a chromatography resin in accordance with the present invention can be achieved by conventional means known to those of skill in the art. Examples of proteins include but are not limited to antibodies, enzymes, growth regulators, clotting factors, transcription factors and phosphoproteins. In many such conventional procedures, the chromatography resin prior to use is equilibrated with a buffer at the pH that will be used for the binding of the target protein (e.g., an antibody or a non-antibody protein). Equilibration can be done with respect to all features that will affect the binding environment, including ionic strength and conductivity when appropriate.

In some embodiments, the chromatography resins described herein can be used in “bind-elute” mode to purify a target protein from a biological sample. In some embodiments, following binding of the target protein to the chromatography resin, a change in pH can be used to elute the target protein.

In some embodiments, once the chromatography resin is equilibrated, a sample containing the target protein (e.g., a biological sample) is loaded onto the chromatography resin. The sample is maintained at a pH of between about 4.5 and about 8 with an appropriate buffer, allowing the target protein to bind to the chromatography resin. Notably, it has been found that the mixed mode chromatography resins described herein function with solutions having salt concentrations in the range of salt concentrations of cell cultures (e.g., 50-300 mM, or about 100-150 mM). Thus, in some embodiments, the protein is loaded to the chromatography resin under such salt concentrations.

In some embodiments, the chromatography resin is then washed with a wash buffer, optionally at the same pH as that of the loading step, to remove any proteins that may have been present in the source liquid. The bound target protein (e.g., antibody or non-antibody protein, as desired) can be subsequently eluted. In some embodiments, the protein is then eluted with an elution buffer at a pH above about 4.5, about 5.0, about 6.0, or about 7.0. Illustrative pH ranges, as cited above, are a pH of about 4.5 to about 8 for the binding and washing steps, and pH of about 4.5 to about 8, about 5.0 to about 8.0, about 6.0 to about 8.0, or about 7.0 to about 8.0 for the elution step. In certain embodiments, the binding and washing steps are performed with the inclusion of a salt in the sample and wash liquids. Examples of salts that can be used for this purpose are alkali metal and alkaline earth metal halides, notably sodium and potassium halides, and as a specific example sodium chloride. The concentration of the salt can vary; in most cases, an appropriate concentration will be one within the range of about 10 mM to about 1.5M. As will be seen in the working examples below, optimal elution conditions for some proteins will involve a buffer with a higher salt concentration than that of the binding buffer, and in other cases by a buffer with a lower salt concentration than that of the binding buffer. The optimal choice in any particular case is readily determined by routine experimentation.

The chromatography resin can be utilized in any conventional configuration, including packed columns and fluidized or expanded-bed columns, and by any conventional method, including batchwise modes for loading, washes, and elution, as well as continuous or flow-through modes. The use of a packed flow-through column is particularly convenient, both for preparative-scale extractions and analytical-scale extractions. A column may, thus, range in diameter from 1 cm to 1 m, and in height from 1 cm to 30 cm or more. In some embodiments, a flow-through column can contain a mixture of particles, each particle comprising one of the chromatography ligands disclosed herein. In other embodiments, one or more chromatography ligand can be immobilized on a solid support, such as a particle, membrane or monolith to provide a chromatography resin that provides a mixture of chromatography ligands disposed on the solid support.

### Additional Disclosure and Claimable Subject Matter

1. A ligand of the formula:

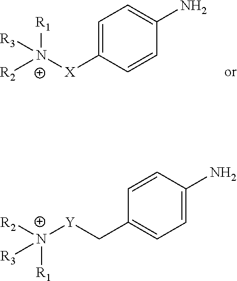

where R₁, R₂, and R₃ can be the same or different and are optionally substituted C₁-C₁₀ alkyl, H, or optionally substituted —(CH₂CH₂O)ₙ—H, where n is 1, 2, 3, 4, or 5, with the proviso that at least one of R₁, R₂, and R₃ is not H when one of R₁, R₂, and R₃ is H; X is —(C₁-C₅ alkyl)- or —(CH₂CH₂O)ₙ—, and n is 1, 2, 3, 4, or 5; and Y is —(C₁-C₈alkyl)- or —(CH₂CH₂O)ₙ— and n is 1, 2, 3, 4, or 5, the oxygen of the ethyloxy group in X or Y being bonded to the benzyl ring when present.

2. The ligand of embodiment 1, wherein R₁, R₂ and R₃ are not substituted and are C₁-C₁₀ alkyl or —(CH₂CH₂O)ₙ—H, where n is 1, 2, 3, 4, or 5.

3. The ligand of embodiment 1 or embodiment 2, wherein R₁, R₂ and R₃ are the same or different and are a C₁-C₈alkyl group.

4. The ligand of embodiments 1-3, wherein R₁, R₂ and R₃ are the same or different and are a C₁-C₃ alkyl group.

5. The ligand of any one of embodiments 1-4, wherein R₁, R₂ and R₃ are the same and are a methyl group or an ethyl group.

6. The ligand of embodiment 1, wherein R₁, R₂ and R₃ are the same or different and are substituted by one, two or three radicals independently selected from C₁-C₁₀ alkyl, a benzyl group, a phenyl group, or a hydroxyl group.

7. The ligand of embodiment 1, wherein R₁, R₂, and/or R₃ are the same or different and are H, C₁-C₅ groups, optionally substituted with one or more hydroxyl group, or C₁-C₃ groups, optionally substituted with one or more hydroxyl group, with the proviso that at least one of R₁, R₂, and R₃ is not H when one of R₁, R₂, and R₃ is H.

8. The ligand of embodiment 7, wherein one of R₁, R₂ or R₃ is —CH₂CH₂OH group and the other two radicals can be the same or different and are a H, C₁-C₁₀ alkyl group, a C₁-C₅ alkyl group, a methyl group, an ethyl group or a propyl group.

9. The ligand of embodiment 8, wherein one of R₁, R₂ or R₃ is —CH₂CH₂OH and the other two radicals are the same and are a methyl or ethyl group.

10. The ligand of embodiment 1, wherein the ligand is CB466x, CB466q, CB464b4, or CB467b.

11. A mixed-mode chromatography medium (chromatography resin) comprising a solid support, linker, and a ligand and having the formula:

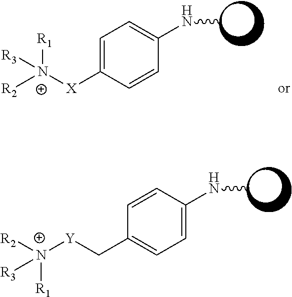

where R₁, R₂, and R₃ can be the same or different and are optionally substituted C₁-C₁₀ alkyl, H, or optionally substituted —(CH₂CH₂O)ₙ—H, where n is 1, 2, 3, 4, or 5, with the proviso that at least one of R₁, R₂, and R₃ is not H when one of R₁, R₂, and R₃ is H; X is —(C₁-C₈alkyl)- or —(CH₂CH₂O)ₙ—, and n is 1, 2, 3, 4, or 5; and Y is —(C₁-C₈alkyl)- or —(CH₂CH₂O)ₙ— and n is 1, 2, 3, 4, or 5, the oxygen of the ethyloxy group in X or Y being bonded to the benzyl ring when present and the linker is an alkyl group.

12. The mixed mode chromatography medium of embodiment 11, wherein R₁, R₂ and R₃ are not substituted and are C₁-C₁₀ alkyl or —(CH₂CH₂O)ₙ—H, where n is 1, 2, 3, 4, or 5.

13. The mixed mode chromatography medium of embodiment 11 or embodiment 12, wherein R₁, R₂ and R₃ are the same or different and are a C₁-C₅ alkyl group.

14. The mixed mode chromatography medium of embodiments 11-13, wherein R₁, R₂ and R₃ are the same or different and are a C₁-C₃ alkyl group.

15. The mixed mode chromatography medium of embodiments 11-14, wherein R₁, R₂ and R₃ are the same and are a methyl group or an ethyl group.

16. The mixed mode chromatography medium of embodiment 11, wherein R₁, R₂ and R₃ are the same or different and are substituted by one, two or three radicals independently selected from C₁-C₁₀ alkyl, a benzyl group, a phenyl group, or a hydroxyl group.

17. The mixed mode chromatography medium of embodiment 11, wherein R₁, R₂, and/or R₃ are the same or different and are H, C₁-C₅ groups, optionally substituted by one or more hydroxyl group, or C₁-C₃ groups, optionally substituted with one or more hydroxyl group, with the proviso that at least one of R₁, R₂, and R₃ is not H when one of R₁, R₂, and R₃ is H.

18. The mixed mode chromatography medium of embodiment 17, wherein one of R₁, R₂ or R₃ is —CH₂CH₂OH group and the other two radicals can be the same or different and are a H, C₁-C₁₀ alkyl group, a C₁-C₅ alkyl group, a methyl group, an ethyl group or a propyl group.

19. The mixed mode chromatography medium of embodiment 18, wherein one of R₁, R₂ or R₃ is —CH₂CH₂OH and the other two radicals are the same and are a methyl or ethyl group.

20. The mixed mode chromatography medium of embodiment 11, wherein the ligand is CB466x, CB466q, CB464b4, or CB467b.

21. The mixed mode chromatography medium of any one of embodiments 11-20, wherein the solid support is a particle, a membrane, or a monolith.

22. A method for purifying a protein from a source solution, said method comprising: (a) contacting said source solution with a mixed-mode chromatography medium comprising a mixed-mode chromatography medium according to any one of embodiments 11-21 and binding said protein; and (b) eluting said protein so bound from said solid support.

23. The method according to embodiment 22, wherein said protein is an antibody.

24 The method according to embodiment 22, wherein said protein is a recombinant protein.

25. The method according to any one of embodiments 21-24, wherein step (a) is performed at a pH of about 4.5 to about 9.0 and step (b) is performed at a pH of about 4.5 to about 9.0.

26. The method according to any one of embodiments 21-25, wherein said source solution contains a salt selected from alkali metal and alkaline earth metal halides at a concentration of from about 10 mM to about 100 mM.

27. The method according to any one of embodiments 21-25, wherein said source solution contains a salt selected from alkali metal and alkaline earth metal halides at a concentration of from about 10 mM to about 50 mM.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Coupling of Ligands to a Solid Support

10 ml of UNOsphere aldehyde was suspended in 20 ml of water at room temperature on a shaker at 200 rpm. About 0.6-1.1 g of ligand chemicals were then added and dissolved for 15 minutes, 0.2 g of sodium cyanoborohydride was added and the reaction was continued for 16 hours. Resins were then washed with 100 ml water.

The table below shows the amount of ligand chemicals added to the coupling reaction:

| Ligand | Amount of addition (g) |
|---|---|
| CB 466x | 0.65 |
| CB 466q | 0.60 |
| CB 464b4 | 0.60 |
| CB 467b | 1.10 |

### Example 2—General Procedure for Protein Separation

A 2.2 mL column was packed with each of the immobilized ligands. Each column was equilibrated with Buffer A (20 mM TRIS-HCl (pH=8.5)). Column elution was performed using a linear gradient of Buffer A to Buffer B (20 mM TRIS-HCl at pH=8.5+1M NaCl). Each column was loaded with 250 μL of Bio-Rad's Anion Protein Standard in Buffer A. This protein standard elutes in the order of equine myoglobin, conalbumin, chicken ovalbumin, and soybean trypsin inhibitor.

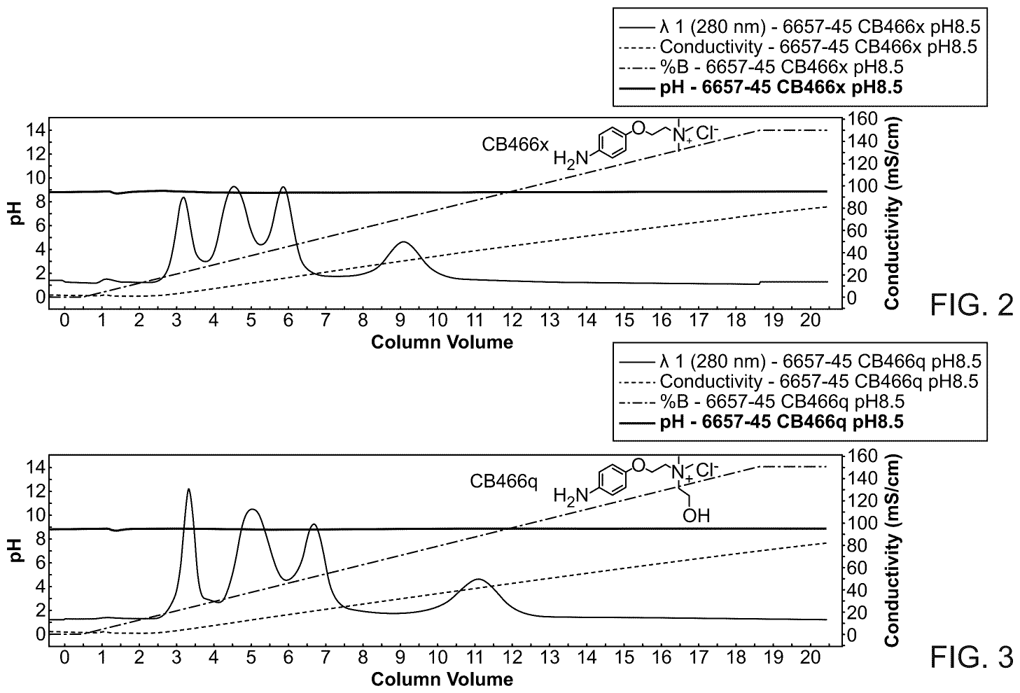

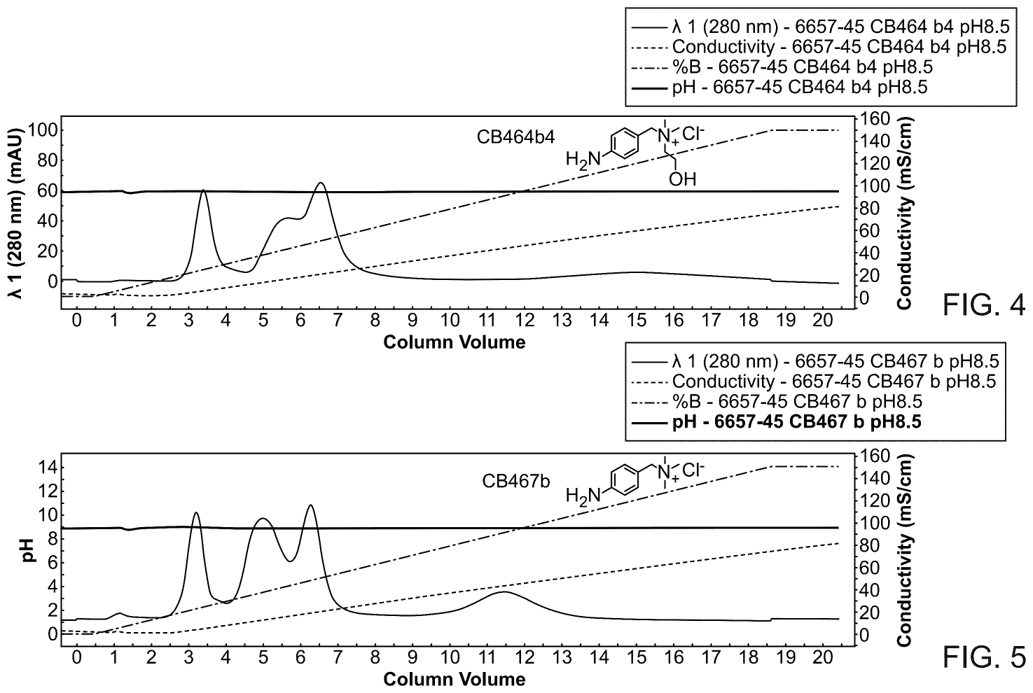

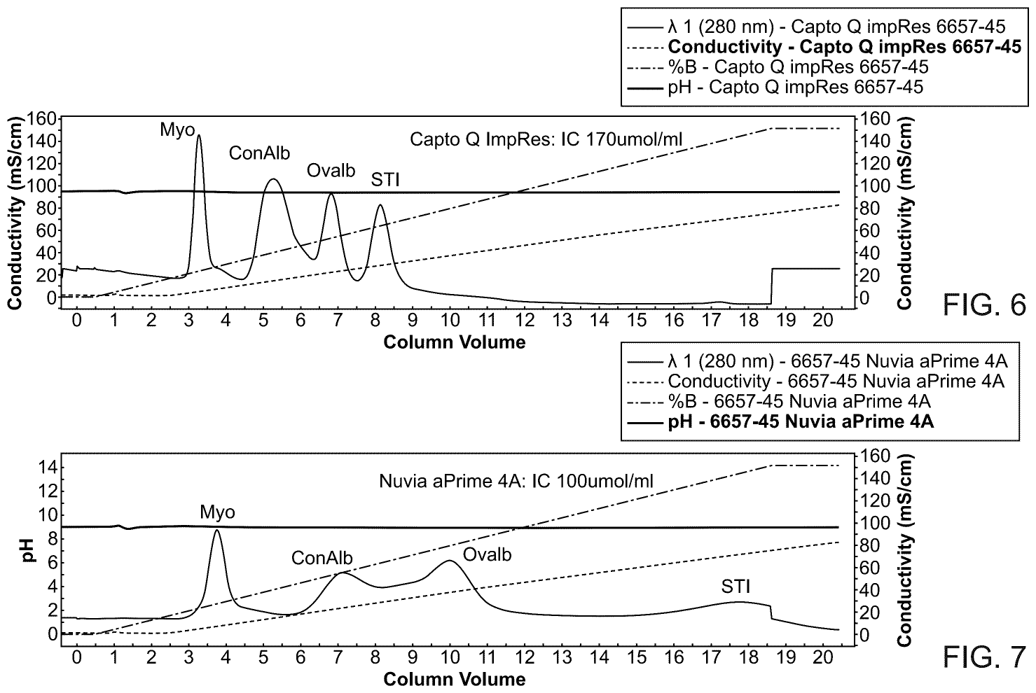

The chromatograph for each respective ligand is provided in FIGS. 2-5. The chromatographs for two commercially available chromatography resins are provided in FIG. 6 (Capto Q ImpRes, Cytiva Life Sciences, Boston MA) and FIG. 7 (Nuvia aPrime 4A (Bio-Rad Laboratories, Inc., Hercules, CA).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

We claim:

1. A ligand of the formula: 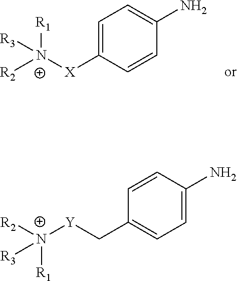
where R₁, R₂, and R₃ can be the same or different and are optionally substituted C₁-C₁₀ alkyl, H, or optionally substituted —(CH₂CH₂O)ₙ—H, where n is 1, 2, 3, 4, or 5, with the proviso that at least one of R₁, R₂, and R₃ is not H when one of R₁, R₂, and R₃ is H; X is —(C₁-C₈alkyl)- or —(CH₂CH₂O)ₙ—, and n is 1, 2, 3, 4, or 5; and Y is —(C₁-C₈alkyl)- or —(CH₂CH₂O)ₙ— and n is 1, 2, 3, 4, or 5, the oxygen of the ethyloxy group in X or Y being bonded to the benzyl ring when present.

2. The ligand of claim 1, wherein R₁, R₂ and R₃ are not substituted and are C₁-C₁₀ alkyl or —(CH₂CH₂O)ₙ—H, where n is 1, 2, 3, 4, or 5.

3. The ligand of claim 1, wherein R₁, R₂ and R₃ are the same or different and are a C₁-C₅ alkyl group.

4. The ligand of claim 2, wherein R₁, R₂ and R₃ are the same or different and are a C₁-C₃ alkyl group.

5. The ligand of claim 2, wherein R₁, R₂ and R₃ are the same and are a methyl group or an ethyl group.

6. The ligand of claim 1, wherein R₁, R₂ and R₃ are the same or different and are substituted by one, two or three radicals independently selected from C₁-C₁₀ alkyl, a benzyl group, a phenyl group, or a hydroxyl group.

7. The ligand of claim 1, wherein R₁, R₂, and/or R₃ are the same or different and are H, C₁-C₅ groups, optionally substituted with one or more hydroxyl group, or C₁-C₃ groups, optionally substituted with one or more hydroxyl group, with the proviso that at least one of R₁, R₂, and R₃ is not H when one of R₁, R₂, and R₃ is H.

8. The ligand of claim 7, wherein one of R₁, R₂ or R₃ is —CH₂CH₂OH group and the other two radicals can be the same or different and are a H, C₁-C₁₀ alkyl group, a C₁-C₅ alkyl group, a methyl group, an ethyl group or a propyl group.

9. The ligand of claim 8, wherein one of R₁, R₂ or R₃ is —CH₂CH₂OH and the other two radicals are the same and are a methyl or ethyl group.

10. The ligand of claim 1, wherein the ligand is CB466x, CB466q, CB464b4, or CB467b.

11. A mixed-mode chromatography medium comprising a solid support, linker, and a ligand and having the formula: 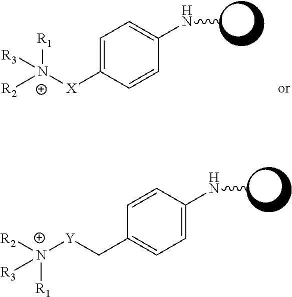
where R₁, R₂, and R₃ can be the same or different and are optionally substituted C₁-C₁₀ alkyl, H, or optionally substituted —(CH₂CH₂O)ₙ—H, where n is 1, 2, 3, 4, or 5, with the proviso that at least one of R₁, R₂, and R₃ is not H when one of R₁, R₂, and R₃ is H; X is —(C₁-C₅ alkyl)- or —(CH₂CH₂O)ₙ—, and n is 1, 2, 3, 4, or 5; and Y is —(C₁-C₈alkyl)- or —(CH₂CH₂O)ₙ— and n is 1, 2, 3, 4, or 5, the oxygen of the ethyloxy group in X or Y being bonded to the benzyl ring when present and the linker is an alkyl group.

12. The mixed mode chromatography medium of claim 11, wherein R₁, R₂ and R₃ are not substituted and are C₁-C₁₀ alkyl or —(CH₂CH₂O)ₙ—H, where n is 1, 2, 3, 4, or 5.

13. The mixed mode chromatography medium of claim 12, wherein R₁, R₂ and R₃ are the same or different and are a C₁-C₈alkyl group or a C₁-C₃ alkyl group.

14. The mixed mode chromatography medium of claim 13, wherein R₁, R₂ and R₃ are the same and are a methyl group or an ethyl group.

15. The mixed mode chromatography medium of claim 11, wherein R₁, R₂ and R₃ are the same or different and are substituted by one, two or three radicals independently selected from C₁-C₁₀ alkyl, a benzyl group, a phenyl group, or a hydroxyl group.

16. The mixed mode chromatography medium of claim 11, wherein R₁, R₂, and/or R₃ are the same or different and are H, C₁-C₅ groups, optionally substituted by one or more hydroxyl group, or C₁-C₃ groups, optionally substituted with one or more hydroxyl group, with the proviso that at least one of R₁, R₂, and R₃ is not H when one of R₁, R₂, and R₃ is H.

17. The mixed mode chromatography medium of claim 16, wherein one of R₁, R₂ or R₃ is —CH₂CH₂OH group and the other two radicals can be the same or different and are a H, C₁-C₁₀ alkyl group, a C₁-C₅ alkyl group, a methyl group, an ethyl group or a propyl group.

18. The mixed mode chromatography medium of claim 17, wherein one of R₁, R₂ or R₃ is —CH₂CH₂OH and the other two radicals are the same and are a methyl or ethyl group.

19. The mixed mode chromatography medium of claim 11, wherein the ligand is CB466x, CB466q, CB464b4, or CB467b.

20. A method for purifying a protein from a source solution, said method comprising: (a) contacting said source solution with a mixed-mode chromatography medium comprising a mixed-mode chromatography medium according to claim 11 and binding said protein; and (b) eluting said protein so bound from said solid support.
